(19) United States
(12) Patent Application Publication
(10) Pub. No.: US 2012/0201786 A1
(43) Pub. Date: Aug. 9, 2012

(54) METHODS FOR THE USE OF STEM CELLS AND STEM CELL FACTORS IN THE TREATMENT OF SKIN CONDITIONS

(76) Inventor: Nikolai Tankovich, San Diego, CA (US)

(21) Appl. No.: 13/184,451

(22) Filed: Jul. 15, 2011

Related U.S. Application Data
(60) Provisional application No. 61/364,783, filed on Jul. 15, 2010.

(51) Int. Cl. A61K 35/12; A61P 17/00; A61K 35/36; A61K 35/28; A61K 35/14

(52) U.S. Cl. 424/93.7

(57) ABSTRACT

The invention provides a method for treating a skin condition in a patient through the administration of stem cells, stem cell factors or a combination thereof. The invention finds use in medical (i.e. healing) and cosmetic applications. Disclosed herein are methods for making and using compositions of stem cells and stem cell factors, and methods for their use in the treatment of skin conditions.

This application claims priority to provisional application Ser. No. 61/364,783 filed Jul. 15, 2010, the contents of which are incorporated by reference in their entirety.

The present invention is in the field of stem cell therapy. In particular, the invention relates to methods of using stem cell therapy in medical and cosmetic applications.

### BACKGROUND

Skin is subject to insults by many extrinsic and intrinsic factors. Extrinsic factors that can adversely affect the skin include wounds, ultraviolet radiation (e.g., from sun exposure), environmental pollution, wind, heat, low humidity, harsh surfactants, abrasives, and the like. Intrinsic factors that lead to skin problems include chronological aging, biochemical changes from within the skin, disease and medical disorders. Whether extrinsic or intrinsic, these factors result in visible signs of skin aging and damage, such as wrinkling, roughness and histological changes. As a result, the improvement of the appearance and health of the skin has become a concern for many people.

Extrinsic or intrinsic factors may result in the thinning and general degradation of the skin. For example, as the skin naturally ages, there is a reduction in the cells and the blood vessels that supply the skin. There is also a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990, which is incorporated by reference herein in its entirety. Moreover, the epidermis has a cell renewal system in which fully differentiated cells are continually shed from the skin surface and replaced by new cells formed within the germative cell layers of the skin. With increasing age, cell renewal rates decrease, leading to the development of coarse, sallow skin.

Skin also contains an elaborate network of elastin fibers that is responsible for maintaining its elastic properties. With excessive exposure to sunlight the elastic fiber system becomes hyperplastic, disorganized and ultimately disrupted. This process is known as actinic elastosis and it is a principal cause of wrinkling, discoloration and laxity of the skin in the exposed areas of the body. As new fibroblasts, endothelial cells and keratinocytes form, the skin can repair itself. However, the skin becomes less able to do so as it ages. Therefore, agents that can accelerate the growth and repair of prematurely aged skin are needed. Wound healing may also be accelerated by increased cellular proliferation and migration of certain cell types. The mechanisms involved in wound healing are often divided into four phases: hemostasis, inflammation, proliferation and maturation. During inflammation, leucocytes accumulate to combat bacteria and the permeability of blood vessel walls increases, leading to swelling. If an infection does not develop the number of leucocytes diminishes. Monocytes replace the leukocytes. Macrophages and lymphocytes release growth factors (cytokines) as well as a number of chemical substances, such as histamine, serotonin, and prostaglandins. These substances help regulate the wound healing process. In the proliferation phase, new fibroblasts, endothelial cells and keratinocytes arise, connective tissue is formed, new blood vessels grow and injured tissue is regenerated. Fibroblasts become dominant after about a week, the inflammation decreases and the strength of the tissues around the wound site increases rapidly. During the maturation phase collagen is laid down and scar tissue is formed. This maturation phase might go on for a long time, during which time, tissues of various types are regenerated. In order to obtain an optimal healing of skin and associated tissues, the supply of different vitamins and trace elements as well as nutrients should be sufficient as well as the oxygen supply.

Anti-wrinkle treatments range from cosmetic creams and moisturizers to various forms of cosmetic surgery. However, while certain skin care compositions are available on the market, such compositions do not effectively stimulate the healing, growth, turnover and overall health of new skin tissues. Hence, there is a need for a skin therapy that not only improves the appearance but also the health of skin. In addition, such treatments would ideally provide a range of useful activities such as, for example, wound closure, wound healing, skin renewal, skin rejuvenation, scar reduction, soothing rashes, eliminating wrinkles and reducing the signs of aging.

### SUMMARY OF THE INVENTION

An objective of the invention is to provide a method for treating a skin condition in a patient through the administration of stem cells, stem cell factors or a combination thereof. The invention finds use in medical (i.e. healing) and cosmetic applications. Disclosed herein are methods for making and using compositions of stem cells and stem cell factors in the treatment of skin conditions.

### BRIEF DESCRIPTION OF THE DRAWINGS

FIG. 1 is a microscopic image of a primary bone marrow cell suspension.

FIGS. 2 and 3 depict microscopic images of primary human bone marrow cell cultures at 4 and 10 days respectively.

FIGS. 4a-f are graphs showing the expression profiles of human mesenchymal stem cells for CD105, CD90, CD44, CD34, HLA-ABC and HLA-DR respectively

### DEFINITIONS

The term “mesenchymal cell” refers to mesodermal germ lineage cells which may or may not be differentiated. The mesenchymal cells of the invention include cells at all stages of differentiation beginning with multipotent mesenchymal stem cells, down to fully differentiated terminal cells.

The term “ectodermal cells” refers to ectodermal germ lineage cells which may or may not be differentiated. The ectodermal cells of the invention include cells at all stages of differentiation beginning with multipotent ectodermal stem cells, down to fully differentiated terminal cells.

The term “endodermal cells” refers to endodermal germ lineage cells which may or may not be differentiated. The endodermal cells of the invention include cells at all stages of differentiation beginning with multipotent endodermal stem cells, down to fully differentiated terminal cells.

The term “patient,” or “subject,” refers to animals, including mammals, preferably humans, who are treated with the pharmaceutical compositions or in accordance with the methods described herein

The term “pharmaceutically acceptable carrier” (or medium), which may be used interchangeably with the term “biologically compatible carrier” (or medium), refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, a “therapeutically effective amount,” or “effective amount,” refers to the number of transplanted cells which are required to produce a therapeutic effect or improvement in the disorder (e.g. skin condition) that is being treated.

The term “stem cell composition” refers to a composition of whole cell stem cells, a composition of stem cell factors, or a combination of whole cell stem cells and stem cell factors. The composition optionally comprises purified stem cell components. The stem cells for the composition may be derived from any stem cell source including embryonic stem cells, prenatal stem cells, adult stem cells, hematopoietic stem cells and fetal cord blood stem cells. One skilled in the art will appreciate that the stem cell composition may be derived from any other stem cell source known in the art at the time this application was filed.

“Rejuvenating composition” refers to a composition that improves the appearance of, or rectifies, a condition (e.g. skin condition).

As used herein, “treating” or “treat” includes (i) preventing a pathologic condition (e.g. skin condition) from occurring; (ii) inhibiting the pathologic condition (e.g. skin condition) or arresting its development; (iii) relieving the pathologic condition (e.g. skin condition), or (iv) alleviating the symptoms associated with the condition (e.g. skin condition).

The term “clone,” or “clonal cell,” refers to a single cell which is expanded to produce an isolated population of phenotypically similar cells (i.e. a “clonal cell population”).

The term “cell line” refers to one or more generations of cells which are derived from a clonal cell.

The terms “administration” and “administering” as used herein refer to the delivery of a stem cell composition by an administration route including, but not limited to, intravenous, intra-arterial, intramuscular, intraperitoneal, subcutaneous, intramuscular, topically, or combinations thereof.

### DETAILED DESCRIPTION

The invention relates to a method of using stem cell therapy in the treatment of skin disorders. The invention generally involves obtaining a composition of therapeutic stem cells and/or stem cell factors, and administering the composition to a patient suffering from a selected skin disorder.

The invention may be practiced with any stem cell composition that treats a skin condition when administered as disclosed herein. In some aspects of the invention, the stem cell composition comprises stem cells in a pharmaceutically acceptable carrier. In other aspects of the invention, the stem cell composition comprises stem cells and stem cell factors in a pharmaceutically acceptable carrier. In yet other aspects of the invention, the stem cell composition comprises stem cell factors in a pharmaceutically acceptable carrier. As used herein, the term “stem cell” refers to an undifferentiated cell which has the ability to both self-renew (through mitotic cell division) and undergo differentiation to form a more specialized cell. Stem cells have varying degrees of potency. A precursor cell is but one example of a stem cell.

Stem cells for use with the invention include pluripotent stem cells which are capable of forming cells from each of the mesoderm, endoderm and ectoderm germ layers. In other aspects of the invention, the stem cell composition comprises multipotent stem cells, which may include mesnechymal stem cells, ectodermal stem cells, endodermal stem cells, and combinations thereof.

Mesenchymal stem cells for use with the invention may be derived from any human or non-human tissue that provides a therapeutic effect when administered as disclosed herein. Suitable tissue sources include prenatal sources, postnatal sources, and combinations thereof. Tissues for deriving a suitable source of mesenchymal stem cells include, but are not limited to, bone marrow, blood (peripheral blood), dermis (e.g. dermal papillae), periosteum, synovium, peripheral blood, skin, hair root, muscle, uterine endometrium, adipose, placenta, menstrual discharge, chorionic villus, amniotic fluid and umbilical cord blood. Mesechymal stem cells may be derived from these sources individually, or the sources may be combined (before or after enrichment) to produce a mixed population of mesenchymal stem cells from different tissue sources.

Mesenchymal stem cell compositions for use with the invention may comprise purified or non-purified mesenchymal stem cells. Mesenchymal stem cells for use with the invention, and their methods of manufacture, are disclosed in the following references, the disclosures of which are incorporated herein by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

In some aspects of the invention, stem cells for use with the invention are grown (i.e. cultured) under low oxygen conditions. Without being limited to any particular theory, culturing the stem cells under low oxygen conditions increases stem cell proliferation and enhances the production of stem cell factors beneficial in the regeneration of tissues in vivo.

The term “low oxygen” as used herein refers to the cell culture conditions in which stem cells are grown in an environment (i.e. incubator) having reduced oxygen tension (i.e. any oxygen concentration that is less than atmospheric oxygen). Thus, the stem cells for use with the invention may be grown in an oxygen concentration that is below about 20%, preferably below about 15%, more preferably below about 5-10%, at sea level. Low oxygen conditions may be kept as close as possible to the normal physiological oxygen conditions in which a particular stem cell would be found in vivo.

In one embodiment, the low oxygen conditions comprise an ambient (e.g. incubator) oxygen condition of between about 0.25% to about 18% oxygen. In another embodiment, the ambient oxygen conditions comprise between about 0.5% to about 15% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures

Methods for manufacturing stem cells under low oxygen conditions as disclosed herein are available in the art, including the methods disclosed in the following publications, the disclosures of which are incorporated herein by reference. U.S. Pat. No. 6,759,242; U.S. Pat. No. 6,846,641; U.S. Pat. No. 6,610,540; J. Cereb. Blood Flow Metab. 2008 Sep. 28(9):1530-42; Stem Cells. 2008 May 26(5):1325-36; Exp Neurol. 2008 April 210(2):656-70; Mol. Cell. Neurosci. (2007), doi:10.1016/j.mcn.2007.04.003; Experimental Neurology 170, 317-325 (2001); and Neurosignals 2006-07, 15:259-265. Although these references disclose particular procedures and reagents, any low oxygen culture condition capable of expanding stem cells according to the invention may be used.

Skin conditions may be treated, for example, by administering a composition comprising stem cell factors, or stem cell factors in combination with stem cells. The term “stem cell factor” refers to cellular molecules which influence the growth, proliferation, commitment, and differentiation, for example, of other cells (e.g. stem cells) either in vivo or in vitro. Stem cell factors include, but are not limited to, cytokines, growth factors, common beta chain, common gamma chain, and IL-6 cytokine families, adrenomedullin, insulin-like growth factor, epidermal growth factor EGF, fibroblast growth factor FGF, autocrin motility factor, GDF, IGF, PDGF, VEGFA, growth differentiation factor 9, erythropoietin, activins, TGF-α, TGF-β, bone morphogenetic proteins (BMPs), Hedgehog molecules, Wnt-related molecules, and combinations thereof.

The invention finds use in medical and cosmetic applications for improving the health and appearance of the skin. Thus, the invention finds use in the treatment of degenerative skin conditions due to aging including, but not limited to, loss of skin elasticity (e.g. elastosis, solar elastosis, wrinkles and lines), loss of skin pigmentation (e.g. hypopigmentation, liver spots and lentigos), loss of skin turgor and decreased skin thickness.

The term “skin condition” also refers to disorders of the skin including, but not limited to, degenerative skin condition, skin disease, skin problems, which include, but are not limited, acne, eczema, psoriasis, rosacea, vitiligo, leucoderma, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis. In one specific embodiment, the skin disorder can be mediated by an immunological response. In another specific embodiment, the skin disorder can be a lymphocyte-mediated skin disorder. In another specific embodiment, the skin disorder can be selected from the group of alopecia greata, psoriasis, atopic dermatitis, lupus erythematosis, bullous pemphigoid, and psoriatic plaque. In another specific embodiment, the skin disorder can be psoriasis. In another specific embodiment, the skin disorder can be a chronic skin disorder. In another specific embodiment, the skin disorder can be an autoimmune skin disorder. In another specific embodiment, the skin disorder can be a malignant lymphoid disease that manifests in the skin.

The invention may also be applied in the treatment of injuries to the skin. That is, the invention may be used to improve the cosmetic appearance of, for example, scars, including burns (e.g. chemical and thermal burns). The invention also finds application in the treatment of vascular disorders of the skin such as varicose veins, chronic (long-term) venous insufficiency, thrombophlebitis, and arteriovenous fistula.

As noted above, the invention is practiced by administering a stem cell composition to a patient suffering from a skin condition. As used herein, the terms “administering,” “administered” and “administer” refer to any administration route by which a stem cell composition can be administered to a patient for a therapeutic effect as disclosed herein. For example, the stem cell composition may be administered intravenously, intra-arterially, intramuscularly, intraperitoneally, subcutaneously, intramuscularly, intranasaly, sublingually, or a by combination thereof. In a preferred embodiment, the stem cell composition is administered intravenously. In other aspects of the invention, the stem cell composition may be administered locally. For example, elastosis may be treated by subcutaneously administering a stem cell composition where a cosmetic improvement in the skin is desired.

In a specific, non-limiting example of the invention, a stem cell composition is administered in the treatment of elastosis. Such exemplary methods may be practiced by obtaining a composition of mesenchymal stem cells from the bone marrow of an allogeneic donor. The stem cells are preferentially expanded and selected as set forth in Example 2, and prepared for administration using a generally accepted pharmaceutical carrier such as PBS. The dosage of stem cells may range between about 1×10⁶ to about 150×10⁶ stem cells. The stem cell composition is then administered intravenously.

The following examples and any specific embodiments referenced above are offered by way of illustration only. One skilled in the art will appreciate that the invention can encompass other embodiments that are obvious in view of the present disclosure.

### Example 1

### Culture of Mesenchymal Stem Cells

Characteristics and Transport of the Donor Material

The source for the preparation of human MMSC was a bone marrow suspension (BMS) obtained by puncture of the iliac crest.

After the mandatory clinical, laboratory, and instrumental examinations of the patient (for autotransplantation) were performed, including:

1. The filling out of the medical history with an attachment of copies of all discharges from the medical history during previous stages of treatment and examination

2. Complete clinical blood tests

3. Complete blood biochemistry panel, with determination of renin, aldosterone, and brain natriuretic peptide

4. Blood group, Rhesus factor

5. Blood test for HIV and Wasserman test

6. Blood test for hepatitis B and C markers

7. Complete immune status

8. Chest x-ray

9. Ultrasound of abdominal organs, kidneys

10. ECG, Halter monitoring

11. Electrocardiography (rest, exercise)

12. Myocardial scintigraphy

13. Coronarography

BMS was harvested from the posterior iliac crest. The material was harvested in a procedures room with the necessary assortment of surgical and anesthesiology instruments. Exfusion of BMS was performed in accordance with the approved methodology, Instructions for the Preparation of Autologous Bone Marrow from Patients for Clinical Use—Ministry of Health, No. 14/2 of 8 Jan. 1980, and procedural recommendations “Transplantation of Bone Marrow in Acute Radiation Disease in Humans”—Ministry of Health of 3 Nov. 1986

After the skin was treated with iodine-containing solutions, in the area of the posterior crest, a puncture was made through the skin and subcutaneous fatty tissue, through which aspiration needles were inserted. After this, the cortical plate of the iliac crest was pierced and the bone marrow was aspirated from the spongy substance of the bone. To collect 20-50 mL of bone marrow, several punctures of the bone cortical plate were made. This required the skin and subcutaneous fatty tissue to be moved aside using the aspiration needle. (The classic technology requires the aspiration of bone marrow in small batches from each puncture (3-5 mL in a 20-mL syringe); nevertheless, the amount of BMS, extracted from each puncture, can reach 20-50 mL, if the bone marrow flow is good.) Following aspiration, the bone marrow preparation was transferred to a polymer container with anticoagulant.

After harvesting was completed a bandage was applied where the skin was punctured and the BMS was immediately sent to the laboratory for further processing. The amount of BMS collected was 20-100 mL.

The BMS was transported to the laboratory in a sterile polymer container containing anticoagulant (heparin). Transportation of the BMS was carried out with strict observance of aseptic and temperature conditions: the container with the bone marrow suspension was placed in a hermetically sealing isothermal container for transport (+2 to +4° C.). Transportation of the BMS should not exceed 2 hours.

The bone marrow suspension received by the laboratory was tested for infectious agents (by PCR or serological/bacteriological tests). The sample was found to be negative for: HIV-1 and -2; HPV-I and II; HBV; HCV; CMV; HSV-1 and 2; toxoplasma gondii; mycoplasma; Epstein-Barr virus; ureaplasma; Chlamydia; treponema pallidum; enterococci; candida species; aspergillus species; e. coli; staphylococci; streptococci and neisseria gonorrhoeae.

The work with BMS in the laboratory is performed in accordance with the recommendations “Instructions for Controlling the Sterility of Stored Blood, Its Components, Preparations for Preserved Bone Marrow, Blood Substitutes, and Preservation Solutions”—Ministry of Health No. 4-42-4-85 of 17 Sep. 1985.

In accordance with technological regulations, the cell phenotype is monitored for specific, satellite, and negative markers at all stages of the cell transplant preparation, and the contamination test is performed in accordance with the approved cell culture certificate.

Preparation of Fractions of Nucleated Cells from Human Bone Marrow Suspension

Plasma, extraneous material (bone fragments, fat), and erythrocytes were removed from the BMS aspirate.
2.1. An equal volume of PBS solution was added to the BMS aspirate.
2.2. The mixture was added over a Ficoll-Pague solution (Pharmacia) and centrifuged at 400 g for 30 minutes at 10° C.
2.3. The middle fraction of nucleated cells was collected, washed with PBS, and centrifuged at 200 g for 10 minutes.
2.4. The fraction was then resuspended in a hypotonic buffer solution for final elimination of erythrocytes and centrifuged. The hemolyzed supernatant was removed.

The obtained suspension of nucleated cells was plated in plastic dishes in growth medium DMEM/F12 (1/1) (Gibco, Grand Island), containing 20% fetal calf serum (HyClone, USA), 2 mM glutamine, and antibiotics. The plating density of the primary cell suspension was 500,000-1,000,000 cells/cm² on average. Cells were cultured under standard conditions (at 37° C. in an atmosphere of 5% CO₂). After a day, unattached cells were removed, and attached cells were incubated to 70-80% confluence, which generally takes from 10 to 20 days. The culture medium was replaced every 3 days.

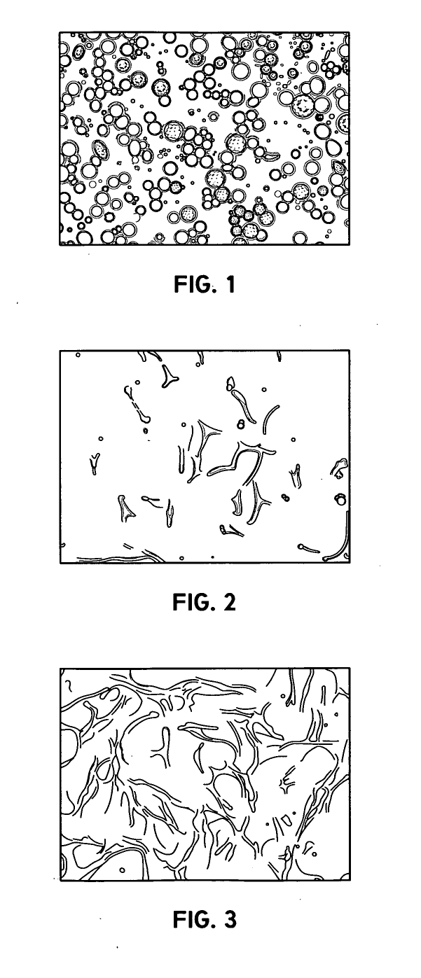

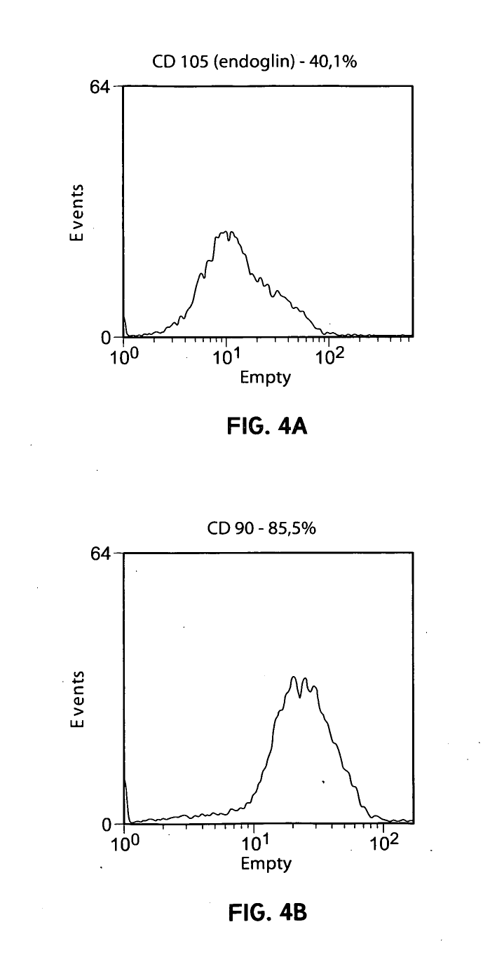

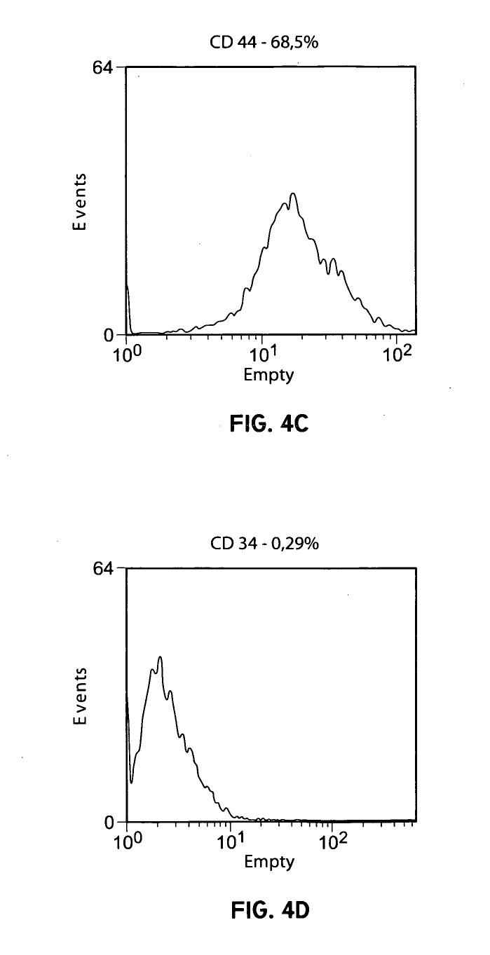

FIG. 1 is an image of the primary cell suspension obtained from the bone marrow aspirate, while FIGS. 2 and 3 depict the primary bone marrow cell culture at 4 and 10 days respectively

Preparation of Cultures, Enriched with Multipotent Mesenchymal Stromal Cells (MMSC)

To select stem populations, MMSC cultures are serially cloned at a low density
3.1. For this purpose, the condensed medium was removed from petri dishes with the primary monolayer culture, reaching 80% confluence, using a 10-mL sterile plastic pipette.
3.2. The petri dishes were washed three times with Versene solution using a 10-mL pipette; then using a 5-mL sterile plastic pipette 2-3 mL of trypsin solution (0.25%) was added, and the dishes were incubated at 37° C., 5% CO₂ for 5-7 minutes.
3.3. The suspension obtained after incubation was homogenized using a 10-mL sterile plastic pipette or Pipetman with a 1-mL tip.
3.4. Up to 10 mL of growth medium was added to the suspension and this was pipetted with a 10-mL sterile plastic pipette until a homogeneous suspension was obtained.
3.5. The number of cells in the obtained suspension was counted using a Goryaev chamber.
3.6. The material was replated in new dishes at a density of 3-4 cells per 1 cm².
3.7. Up to 10 mL of growth medium was added to the petri dishes using a 10-mL sterile plastic pipette.
3.8. The medium was changed every 3 days.

Protocol for Replacing the Growth Medium (Once in 3 Days)
3.8.1 The condensed medium was removed from the petri dish with a 10-mL sterile plastic pipette.
3.8.2. The removed condensed medium was replaced with new medium using a 10-mL sterile plastic pipette in an amount corresponding to the petri dish volume (90 mm-9-10 mL of medium).
3.9. After 10-14 days of culturing with monitoring with an inverted microscope, homogeneous, dense colonies of small cells (7-10 μm in diameter) with a large number of mitoses were selected from dishes, first treated with 1 mM EDTA.
3.10. The colonies were cultured further at a density of 10-50 cells per cm² in the same growth medium at 37° C. in an atmosphere, containing 5% CO₂ and at 95% humidity. The culture medium was replaced every 3 days.
3.11. To reach 50% confluence, the culture was plated at a plating density of 10-50 cells per cm². The number of culture passages did not exceed 5-7.

Protocol for Culture Passaging
3.11.1. The condensed medium was removed from petri dishes with the monolayer culture, reaching 50% confluence, using a 10-mL sterile plastic pipette.
3.11.2. 2-3 mL of trypsin solution was added to the petri dishes using a 5-mL sterile plastic pipette, and the dishes were incubated at 37° C., 5% CO₂ for 5-7 minutes.
3.11.3. The suspension obtained after incubation was homogenized using a 10-mL sterile plastic pipette.
3.11.4. Up to 10 mL of nutrient medium was added to the suspension and this is pipetted with a 10-mL sterile plastic pipette until a homogeneous suspension was obtained.
3.11.5. The cells were counted using a Goryaev chamber.
3.11.6. The cells were then plated into new petri dishes plated at a density of 10-50 cells per cm² using a 10-mL sterile plastic pipette.
3.11.7. Medium was added to the needed volume (to 9-10 mL) to petri dishes using a 10-mL sterile plastic pipette.

Growth medium composition (per 100 mL)

| | | | Measure- | |
|---|---|---|---|---|
| Name | Amount | | ment Units | Manufacturer |
| F12 medium | 49 | | mL | HyClone |
| DMEM medium | 49 | | mL | HyClone |
| Gentamicin 4% | 250 | | μL | Sigma |
| Glutamine | 2 | | mM | PanEko |
| Fibroblast growth | 10 | ng/mL | | ProSpec-Tany |
| factor | | | | TechnoGene LTD |
| Heparin | 8 | U/mL | | |
| FBS FetalClone III | 15 | | mL | HyClone |
| | | | | (SH3010903) |
| Insulin | 1 | μg | mL | |
| Transferrin | 10 | μg | mL | |

### Example 2

### Characterization of Mesenchymal Stem Cells

Cytofluorometric analysis (FACS)—To perform the analysis, during passaging a portion of the cells was suspended in PBS, pH 7.4, after trypsinization at a concentration of 100,000 cells/mL, fixed in 1% methanol at 4° C. for 10 minutes, and then washed. Nonspecific binding was blocked by incubation in 1% BSA and 0.1% goat serum for 1 hour at room temperature. Then, the cells were washed in three volumes of phosphate-buffered saline and centrifuged; the precipitate was suspended in a 0.5% working solution of primary antibodies to 1% BSA with 0.1% goat serum. After incubation for 40 minutes at 4° C., the cells were washed with phosphate-buffered saline, pH 7.4. Mouse monoclonal antibodies (McAb) to CD44, CD90, CD105, CD34, HLA ABC, and HLA DR purchased from PharMingen and Chemicon were used. Nonspecific mouse (rabbit) IgG from the same companies was used as the negative control. Incubation with anti-species antibodies, labeled with FITC or phycoerythrin was performed for 20 minutes. Cells were then washed in phosphate-buffered saline, pH 7.4, and analyzed in a volume of 1 mL in a flow cytofluorometer FACS Calibur (BD Biosciences). Results were analyzed using the program MDI 2.8.

Individual populations were identified in the flow cytofluorometer with use of the combination of McAb for differentiation and activation markers. The number of apoptotic cells was determined using McAb to CD95 (FAS/APO-1 antigen), and the number of hematopoietic cells using McAb to CD34. The functional activity of cellular immunity was evaluated based on the number of cells, expressing the receptor to IL2 (IL2R-CD3+, CD25+) and HLA-DR antigen on their surface, and also based on the number of activated cells (CD71+, CD38+) and activated NK (CD8+, CD16+).

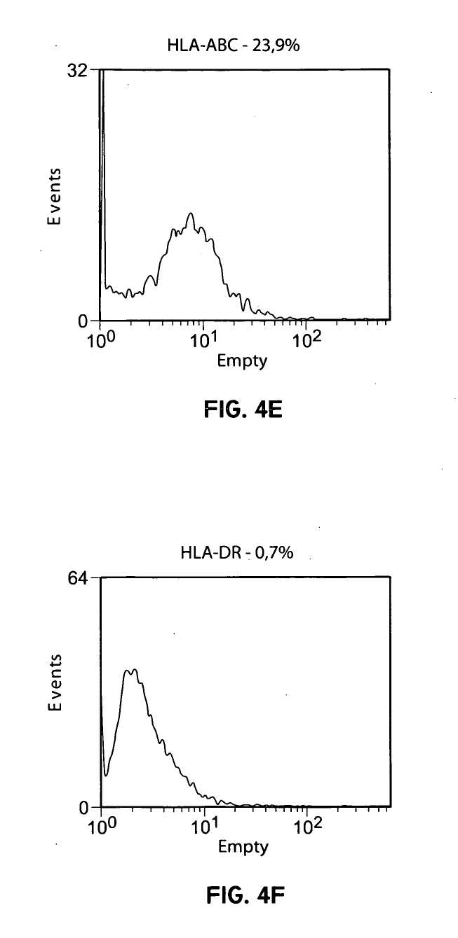

The primary marker of hematopoietic cells (CD34) and HLA DR in clonal cultures MMSC from bone marrow was expressed by less than 1% of cells (at the level of the negative control). The largest cell population (80-92%) was stained by antibodies to CD90 (80-95%), CD44 (60-75%), and endoglin CD105 (about 50%). Antigens MHCl (HLA-ABC) were present on the surface of 5-30% of the cells. The fraction of positive cells changed minimally during passaging, but remained unvaryingly low overall (see FIGS. 4a-f).

### Example 3

### Administration of Cell Composition

Patients of both genders between the ages of 18 were selected for treatment of a skin condition. Mesenchymal stem cells made according to the process of Example 2 were prepared for injection by suspending about 50×10⁶ mesenchymal stem cells in 200 ml of isotonic solution. Mesenchymal stem cells were administered intravenously in a single injection over a 40-60 minute period.

I claim:

1. A method for treating a skin condition in a patient comprising:
a. providing a stem cell composition; and
b. administering the stem cell composition to the patient;
c. wherein administering the stem cell composition treats the skin condition.

2. The method of claim 1, wherein the stem cell composition comprises stem cells, stem cell factors, and a combination thereof.

3. The method of claim 2, wherein the stem cell composition is administered intravenously, intra-arterially, intramuscularly, intraperitoneally, subcutaneously, intramuscularly, or a combination thereof.

4. The method of claim 3, wherein stem cells comprise mesenchymal stem cells, ectodermal stem cells, endodermal stem cells, or a combination thereof.

5. The method of claim 4, wherein the patient has aging skin and the skin condition is selected from elastosis, hypopigmentation, liver spots, hypopigmentation, and combinations thereof.

6. The method of claim 5, wherein the stem cells are mesenchymal stem cells.

7. The method of claim 6, the mesenchymal stem cells are derived from bone marrow, cord blood, hair follicle, and combinations thereof.

8. The method of claim 7, wherein the mesenchymal stem cells are grown under low oxygen conditions.

9. The method of claim 8, wherein the mesenchymal stem cells are grown under low serum conditions.

10. The method of claim 6, wherein the stem cell composition is administered intravenously.

11. A method for treating a skin condition in a patient, wherein the method comprises:
a. providing a stem cell composition;
b. administering an effective amount of the stem cell composition to the patient intravenously;
c. wherein administering the stem cell composition to the patient treats the skin condition.

12. The method of claim 11, wherein the skin condition is selected from elastosis, hypopigmentation, liver spots, hypopigmentation, and combinations thereof.

13. The method of claim 12, wherein the stem cell composition comprises mesenchymal cells, mesenchymal stem cell factors, and combinations thereof.

14. The method of claim 13, wherein the mesenchymal stem cells and mesenchymal stem cell factors are cultured under low oxygen conditions.

15. The method of claim 14, wherein the mesenchymal stem cells are cultured under low oxygen, low serum conditions.
